(19) United States
(12) Patent Application Publication
(10) Pub. No.: US 2015/0306010 A1
(43) Pub. Date: Oct. 29, 2015

(54) PROTECTIVE INGREDIENT OF SKIN BARRIER AND COSMETIC AND/OR DERMATOLOGIC FORMULATION CONTAINING THE SAME

(71) Applicant: BOTICA COMERCIAL FARMACÊUTICA LTDA, São José dos Pinhais (BR)

(72) Inventors: Camila Miranda de Alameda CARVALHO, Curitiba (BR); Israel Henrique Stokfisz FEFFERMAN, Curitiba (BR); Tammy Proença Zagonel NICHELE, Curitiba (BR); Bruna Bastos SWINKA, Curitiba (BR); Alessandro AFORNALI, Curitiba (BR); Carla Abdo BROHEM, Curitiba (BR); Marcela Contador BAPTISTA, Curitiba (BR); Márcio LORENCINI, Curitiba (BR); Gustavo de Campos DIEMANT, Curitiba (BR)

(21) Appl. No.: 14/653,664

(22) PCT Filed: Dec. 20, 2013
(86) PCT No.: PCT/BR2013/000590

§ 371 Date: Jun. 18, 2015

(30) Foreign Application Priority Data
Dec. 21, 2012 (BR) 10 2012 032898 4

(51) Int. Cl. A61K 8/60; A61Q 19/08; A61Q 1/06; A61K 8/14; A61Q 19/10; A61K 8/34; A61Q 17/04

(52) U.S. Cl. CPC A61K 8/60; A61K 8/345; A61Q 19/08; A61Q 17/04; A61K 8/14; A61Q 19/10; A61Q 1/06

(57) ABSTRACT

The present invention describes a skin barrier protective ingredient and a cosmetic and/or dermatological formulation containing the same and which, according to its general features, basically provides a skin barrier protective ingredient from an apple (Malus sp.) extract fraction incorporated into liposomes (soy lecithin) in specific concentrations in order to impart to this liposome-encapsulated apple (Malus sp.) extract fraction a differentiated moisturising activity that protects the skin and minimises the signs of ageing, in that it reinforces and enhances the skin barrier function, induces the proliferation and differentiation of epidermis cells (keratinocytes), develops the epidermis, regulates cell development and differentiation, promotes blood circulation and activates the metabolism of skin ceramides.

The present invention relates to ingredients for cosmetic and/or dermatologic products in general, and more specifically to a skin barrier protective ingredient and a cosmetic and/or dermatologic formulation containing the same which according to their general characteristics has as basic principle to provide a skin barrier protective ingredient from a fraction from the extract of apple Malus sp incorporated into liposomes (soy lecithin) in specific concentrations, in order to provide to such lipossomic fraction of apple Malus sp extract a differentiated moisturizer activity which provides protection to the skin and minimizes the signs of aging through reinforcing and improvement actions of skin barrier, induction of proliferation and differentiation of epidermal cells (keratinocytes), epidermal development, regulation of cell development and differentiation, favoring of blood circulation and activation or the skin ceramide metabolism.

Note that typically the number of divisions that a cell can suffer is limited, and this phenomenon is widely known as Hayflick limit. At each cell division the telomeres are shortened until they reach a critical length when cell proliferation can be stopped and the cell reaches senescence or aging.

The decrease in the regeneration potential of the tissue is one of the main causes of aging. Once the regeneration process of an organism is determined by the proliferative capacity, aging is directly dependent on the proliferation ability of skin cells. Additionally, cell renewal is a known mechanism for maintaining the homeostasis of the skin that involves keratinocyte proliferation and differentiation. With aging, the ability to regenerate the skin decreases. Thus, the stimulation of keratinocyte proliferation is a broad concern approach for the treatment of aging skin, promoting skin barrier enhancement.

In this context, it has become indispensable to structure an asset for skin application in different types of products, for example, soaps, shampoos, lipsticks and makeup in general, sunscreens lotions and creams, indicated primarily for the protection of skin harrier function, minimizing some of the major signs of aging, i.e., enabling to give various kinds of cosmetic and/or dermatologic products with proven application, constituting a novel option for human skin treatment.

At the same time, the demand for all kinds of plant ingredients has grown quite significantly in recent years, focusing on vegetables inputs that perform various functions related to human skin. Mechanisms of action related to the proliferation and differentiation, vitality and skin barrier function are of great interest in the cosmetic and dermatological areas, since they directly reflect the proper functioning and maintenance of the skin tissue of humans.

In this line, the apple extracts have been widely applied for a long time in cosmetic and dermatologic products from the skin care and makeup categories, for example, creams, lotions, soaps, serums, shampoos and sunscreens among many others.

In the specific case, the apple species Malus domestica is widely known in the art for its excellent longevity and ability to remain stable over time. From its tree, a Swiss apple tree known by the name Uttwiler Spütlauber, several portions of tissue were removed in order to obtain plant tissue cultures that allow broad studies of its composition and its properties. In a broad review of the literature in order to establish the current state of art regarding protective ingredients of skin barrier object of the present patent, no relevant documents to the state of the art were disclosed correlating the specific object claimed in the present patent, i.e. a protective cosmetic and/or dermatological ingredient of skin barrier from a fraction derived from apple extract of Malus sp incorporated into liposomes (soy lecithin) in specified concentrations. However, secondary documents were disclosed that fit the references although they do not impede the present patent, in which:

French patent document FR2916972 comprises combining apple extract, apple vinegar, almond glycoproteins, silicone and at least one essential oil. The composition and cosmetic products containing said combination are intended solely to inhibit the action of metalloproteinases, thereby preventing the degradation of elastin fibers in the dermal matrix, which departs from the object claimed in the present patent as it constitutes a distinctive combination of ingredients containing the apple extract.

US patent document US20070036742 comprises the combination of extracts of one or more of the following plants: Boswellia Serrata, Undaria Pinnatifida, green tea, Pureraria Mirifica, liteolin, astilbin, vitamin E, amentoflavone, tetrahydropiperine, licochalcone, astaxanthin, red clover, Brassica Juncea, CoQ10 enzyme, sage, shikimic acid, oleo-resin of hop, apple, soy, ellagic acid, or any derivative, thereof. The composition and cosmetic products containing said combination are intended solely to modulate the growth or regrowth of human hair.

In view of the documents of the state of the art mentioned above, it is clear that it is not provided a fraction from the apple extract Malus domestica, and the cosmetic and/or dermatological formulation containing said fraction is applied in the protection of the skin barrier function, working on specific cell and tissue mechanisms related to the skin, i.e., keratinocyte proliferation activities, improvement of skin barrier function and increased cell metabolism.

Thus, the patent in question is characterized by gathering components in a different design, which will meet the various requirements that the nature of the use demands, impart broad improvement of the skin barrier function providing protection to skin and minimizing some of the main signs of aging, besides acting in increasing the metabolism of lipids. Such design ensures that an ingredient protector of the skin harrier is efficient and reliable due to the aggregate technical qualities which provide advantages and improvements to the maintenance of the general balance of the human skin.

In this conception, the present protective ingredient of skin barrier and the cosmetic and/or dermatological formulation containing the same is based on a fraction from the extract of apple Malus sp, of Swiss origin, capable of acting directly on specific cell and tissue mechanisms related to the aging of human skin, i.e., which ensures to broad anti-aging activity and a perfect protection of the skin harrier function.

More specifically, a fraction from the extract of Malus sp consisting of arabinose, mannose, galactose and inositol defined concentrations was obtained by specific filtration processes, and this fraction was incorporated into a liposome concentration derived from soybean phospholipids (lecithin), so that it became lipossomed, that is, protected by layers of phospholipids that allow its controlled release.

Note that this fraction is closely related to actions of inducing the cell proliferation and improving the skin barrier function, besides acting in increasing the metabolism of lipids, i.e., it is contemplated the offer of a composition containing the fraction obtained from Malus domestica able to act in different cell and tissue mechanisms of the human skin, in addition to having high stability when applied in cosmetic and/or dermatological formulations.

The present invention patent of “Protective Ingredient of Skin Barrier and Cosmetic and/or Dermatologic Formulation Containing the Same” comprises a set of cosmetic and/or dermatological solutions properly embedded, constituting an protective ingredient of skin barrier, the basic formulation of which is consistent with final differentiated qualities that, by their own characteristics, is obtained from a fraction derived from the apple extract of Malus sp. consisting of arabinose, mannose, galactose and inositol in concentrations defined through specific filtration processes, and incorporated into a concentrate of liposomes derived from soybean phospholipids (lecithin) so that it became lipossomed, being the formulation and consequently the amounts employed based directly on the applications to which they are intended in order to allow obtaining an excellent protective ingredient of skin barrier that confers broad and effective overall balance to the skin and mitigates the aging by various cosmetic and/or dermatological formulations.

The “Protective Ingredient of Skin Barrier and Cosmetic and/or Dermatologic Formulation Containing the Same” comprises cosmetic and/or dermatological formulations in which their basic formulations consist of a range of about one hundredth percent to ten percent by weight of the protective ingredient of skin barrier.

The “Protective Ingredient of Skin Barrier and Cosmetic and/or Dermatologic Formulation Containing the Same” comprises cosmetic and/or dermatological formulations wherein said protective ingredient of skin barrier will eventually be added, and it may include ingredients constituent of the base composition or other active or functional ingredients such as surfactants, humectants, thickeners, fragrances, preservatives, sequestrants, sunscreens, scents, dyes, emulsifiers, active ingredients, sensory modifiers among others. Considering the features and benefits described above, tests were carried out by various methodologies for evidence of the benefits and effectiveness of the ingredient of said formulations that contain the protective ingredient of skin barrier abject of the present patent. The protocols used and results related to these parameters, as well as the formulations, are showed as examples in the following.

The cytotoxicity of the apple extract fraction was investigated using the MTS technique (a tetrazole compound), and the evaluation of the mechanism of cell death was by flow cytometry, by quantifying the specific protein markers. These tests indicated the absence of cytotoxicity in human skin fibroblasts until the maximum concentration tested, besides the absence of significant mechanisms of apoptosis or necrosis in the concentrations evaluated. Thus, the absence of processes related to the cytotoxicity to said fraction of apple extract was evidenced.

Afterwards, the effectiveness of the apple extract fraction was investigated in the proliferation of keratinocytes using the technical evaluation of keratinocyte proliferation rate in cell culture, after forty-eight hours in the presence or absence of the apple extract fraction. The proliferative capacity in reconstituted skin was also evaluated to twenty-one days for visualization of the number of layers in the epidermis. The results of biological tests showed that the apple extract fraction induced a significant increase in keratinocyte proliferation of about forty percent. In the reconstituted skin, the condition subject to said fraction had increased from 15±2 to 20±2 epidermal layers. Regarding the proliferation of keratinocytes in vitro, there was a significant increase (p≦0.05) of the number of cells after a contact of forty-eight hours with the fraction. These results demonstrated that the apple extract fraction is capable of inducing an increase in keratinocyte proliferation, enhancing skin barrier function. Such results reinforce the role of the apple extract fraction in maintaining the skin structure.

Additionally, skin fragments ex vivo treated with the apple extract fraction were used for twenty-hour hours for DNA microarray analyzes. The microarray results indicated that six hundred thirty-eight genes were modulated by the apple fraction extract, which are involved in the following biological activities: epidermal differentiation, cell growth, keratinocyte differentiation, development and regulation of cell differentiation metabolism of sphingolipids.

As a non-limiting example of cosmetic and/or dermatological formulations containing the protective ingredient of skin barrier object of the present invention patent, the cosmetic and/or dermatological formulations are presented for protective bases of skin barrier for lipstick or makeup in general, shower gel, facial/body/protective lotion and makeup base through the detailed tables below, in which the components are combined and the quantities represented in percent by weight based on the total weight of the composition.

### Example 1

### Lipstick.

| CATEGORY | INGREDIENT | QUANTITY |
|---|---|---|
| Emollients | Acetylated lanolin alcohol | 20-45% |
| | Low molecular weight fatty | |
| | alcohols | |
| | Silicones | |
| | Vegetable oils | |
| Consistency agents | High molecular weight fatty | 10-25% |
| | alcohols | |
| | Vegetable waxes | |
| | Synthetic waxes | |
| Humectants | Glycols | 0.5-2.5% |
| | Polyethylene glycols | |
| | Derived from sucrose | |
| Antioxidants | Natural (vitamins A and E) | 0.01-1% |
| | Synthetic (BHT, BHA) | |
| Dyes | Natural pigments (iron oxides) | 0.05-5% |
| | Synthetic pigments | |
| Active | Malus Domestica fruit Cell | 0.01-10.0% |
| | Culture Extract (Factor 111) | |
| Sequestrants/ | EDTA Gallates | 0.1-0.8% |
| Chelating agents | | |
| Emulsifiers | Sorbitan derivatives | 1.5-3.5% |
| | Ethoxylated alcohols | |
| | Sucrose esters | |
| | Esters of fatty alcohols | |
| Active | Vitamins | 0.02-2.5% |
| | Moisturizers | |
| | Sunscreens | |
| Preservatives | Benzyl alcohol | 0.01-2% |
| | Parabens | |
| | Phenoxyethanol | |
| Organoleptic agents | Fragrance | 0.02-1.5% |

### Example 2

### Shower Gel

| CATEGORY | INGREDIENT | QUANTITY |
|---|---|---|
| Anionic surfactants | Sulfated fatty alcohols | 20-50% |
| Foaming agents | Sulfated and ethoxylated fatty | |
| | alcohols | |
| | Succinated fatty alcohols | |
| Non-ionic surfactants | Amidated coconut derivatives | 2-10% |
| Foam stabilizer | Ethoxylated coconut derivatives | |
| Consistency donors | | |
| Humectants | Glycols | 0.5-2.5% |
| | Polyethylene glycols | |
| | Derived from sucrose | |
| Antioxidants | Natural (vitamins A and E) | 0.01-1% |
| | Synthetic (BHT, BHA) | |
| Dyes | Synthetic pigments | 0.05-5% |
| Sequestrants/ | EDTA Gallates | 0.1-0.8% |
| Chelating agents | | |
| Amphoteric surfactants | Coconut amphoteric agents | 1.5-10% |
| Foam stabilizer and | Corn amphoteric agents | |
| formulation | | |
| Active | Vitamins | 0.02-2.5% |
| | Moisturizers | |
| | Sunscreens | |
| Preservatives | Benzyl alcohol | 0.01-2% |
| | Parabens | |
| | Phenoxyethanol | |
| Thickeners | Pulyethylene glycols derivatives | 0.2-2% |
| Pearly effect | Medium chain fatty alcohols | |
| Active | Malus Domestica Fruit Cell | 0.01-10.0% |
| | Culture Extract (Factor III) | |
| Thickeners | Carbomers | 0.2-2% |
| | Cellulose derivatives | |
| | Polyacrylamides | |
| Organoleptic agents | Fragrance | 0.02-1.5% |

### Example 3

### Facial Cream/Body/Shield

| CATEGORY | INGREDIENT | QUANTITY |
|---|---|---|
| Vehicle | Demineralized water | q.s. |
| Sequestrants/ | Disodium EDTA and/or others | 0.01-0.2% |
| Chelating agents | | |
| Thickeners | Carbomers | 0.1-2% |
| | Cellulose derivativies | |
| | Polyacrylamides | |
| Humectants | Glycols | 0.2-10% |
| Moisturizers | Polyethylene glycols | |
| Active | Derived from sucrose | |
| | Hyaluronates | |
| | Alpha hydroxy acids | |
| Emulsifiers | Sorbitan derivatives | 0.1-5% |
| | Ethoxylated fatty alcohols | |
| | Sucrose esters | |
| | Esters of fatty alcohols | |
| Consistency agents | High molecular weight fatty | 0.5-5% |
| | alcohols | |
| | Vegetable waxes | |
| | Synthetic waxes | |
| | Vegetable oils | |
| pH adjusters | Triethanolamine | Qs |
| | Aminomethyl propanol | |
| | Sodium hydroxide | |
| Emollients | Acetylated lanolin alcohol | 1-15% |
| | Low molecular weight fatty | |
| | alcohols | |
| | Silicones | |
| | Vegetable oils | |
| | Short and medium chain | |
| | triglycerides | |
| Active | Vegetable extracts | 0.01-10% |
| | Biotechnological extracts | |
| | Vitamins | |
| | Vegetable oils | |
| | Peptides | |
| | Others | |
| Active | Malus Domestica Fruit Cell | 0.01-10.0% |
| | Culture Extract (Factor III) | |
| Organoleptic agents | Fragrance | 0.1-0.5% |
| Preservatives | Phenoxyethanol | 0.2-1.2% |
| | Imidazolidinyl urea | |
| | Others | |

### Example 4

### Base Makeup

| CATEGORY | INGREDIENT | QUANTITY |
|---|---|---|
| Aqueous phase | Carbomers | 0.1-2% |
| thickeners | Cellulose derivatives | |
| | Polyacrylamides | |
| Emollients | Acetylated lanolin alcohol | 2-20% |
| | Low molecular weight fatty | |
| | alcohols | |
| | Silicones | |
| | Vegetable oils | |
| Consistency agents | High molecular weight fatty | 0.1-3% |
| | alcohols | |
| | Vegetable waxes | |
| | Synthetic waxes | |
| Humectants | Glycols | 0.5-10% |
| | Polyethylene glycols | |
| | Derived from sucrose | |
| Antioxidants | Natural (vitamins A and E) | 0.01-1% |
| | Synthetic (BHT, BHA) | |
| Dyes | Natural pigments | 0.01-10% |
| | (iron oxides) | |
| | Synthetic pigments | |
| Sequestrants/ | EDTA Gallates | 0.1-0.8% |
| Chelating agents | | |
| Active | Vegetable extracts | 0.01-10% |
| | Biotechnological extracts | |
| | Vitamins | |
| | Vegetable oils | |
| | Peptides | |
| | Others | |
| Emulsifiers | Sorbitan derivatives | 1.5-3.5% |
| | Ethoxylated fatty alcohols | |
| | Sucrose esters | |
| | Esters of fatty alcohols | |
| Active | Malus Domestica Fruit Cell | 0.01-10.0% |
| | Culture Extract (Factor III) | |
| Active | Vitamins | 0.02-15% |
| | Moisturizers | |
| | Sunscreens | |
| Preservatives | Benzyl alcohol | 0.01-2% |
| | Parabens | |
| | Phenoxyethanol | |
| Organoleptic agents | Fragrance | 0.02-1.5% |

For all the foregoing, it is an protective ingredient of skin barrier that will be well received by the users of cosmetic and/or dermatological formulations in general, because the present protective ingredient of skin barrier and cosmetic and/or dermatological formulation containing the same have numerous advantages, such as: high stability, cost effective, directly applied to a wide range of cosmetic and/or dermatologic products, and be sure to have a protective ingredient of skin barrier and hence the cosmetic and/or dermatological formulations containing the same that meet the current rules and regulations and the basic necessary conditions to its implementation.

All these attributes enable to classify this protective ingredient of skin barrier and cosmetic and/or dermatological formulation containing the same as one versatile, efficient and safe ingredient to be applied in a wide range of cosmetic and/or dermatological formulations, independent of the general characteristics that they can present, not limited at any time to the representations described herein, and that it should be understood in its broader set of claims, i.e., not limited to the particular form disclosed, and that other forms are understood as included within the scope of the appended claims.

What is claimed is:

1. A protective ingredient of skin barrier and cosmetic and/or dermatologic formula containing the same, comprising: a set of cosmetic and/or dermatological solutions constituting a protective ingredient of skin barrier, the basic formulation obtained from a fraction derived from apple extract of Malus sp containing arabinose, mannose, galactose, inositol in specific concentrations and incorporated into a liposome concentrate derived from soy phospholipids (lecithin) so that it became lipossomed, being the formulation and consequently the amounts employed based directly on the intended applications and conferring effective attenuation of aging by various cosmetic and/or dermatological formulations.

2. The protective ingredient of skin barrier and cosmetic and/or dermatologic formula of claim 1, further comprising cosmetic and/or dermatological formulations in which the basic formulations consist of a range of about one hundredth percent to ten percent by weight of the protective ingredient of skin barrier.

3. The protective ingredient of skin barrier and cosmetic and/or dermatologic formula of claim 1, wherein said protective ingredient of skin barrier that will be eventually added may comprises a base composition and other active or functional ingredients selected from the group consisting of surfactants, humectants, thickeners, fragrances, preservatives, sequestrants, sunscreens, scents, dyes, emulsifiers, active ingredients, and sensory modifiers.

4. The protective ingredient of skin barrier and cosmetic and/or dermatologic formula of claim 2, wherein said protective ingredient of skin barrier comprises a base composition and active or functional ingredients selected from the group consisting of surfactants, humectants, thickeners, fragrances, preservatives, sequestrants, sunscreens, scents, dyes, emulsifiers, active ingredients, and sensory modifiers.
